SEQUENCE LISTING (20 sequences)

SEQ ID NO: 1 - DNA, 810 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctctagctcttccaaggctccacccccctcactcccatctcctagtaggctccccggaccatccgacacgcctattctgccccagtag

SEQ ID NO: 2 - protein, 269 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRASSSSKAPPPSLPSPSRLPGPSDTPILPQ

SEQ ID NO: 3 - protein, 148 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRASSSSKAPPPSLPSPSRLPGPSDTPILPQ

SEQ ID NO: 4 - DNA, 1052 bp, Homo sapiens
agagagcgctgggagccggaggggagcgcagcgagttttggccagtggtcgtgcagtccaaggggctggatggcatgctggacccaagctcagctcagcgtccggacccaataacagttttaccaagggagcagctttctatcctggccacactgaggtgcatagcgtaatgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctgacctgccgacacgctccctccccctgccccttctacactctcctgggcccctccctacctcaacctgtaaattattttaaattataaggactgcatggtaatttatagtttatacagttttaaagaatcattatttattaaatttttggaagcataaa

SEQ ID NO: 5 - protein, 241 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 6 - DNA, 1196 bp, Mus musculus
cagcacggcagagagcgcctggagccggaggggagcgcatcgagtgactttggagctggccttatatttggatctcccgggcagctttttggaaactcctagtgaacatgctgtgcctcaagccagtgaaattaggctccctggaggtgggacacgggcagcatggtggagttttggcctgtggtcgtgcagtccagggggctggatggcatgctggacccaagctcacctcagtgtctgggcccaataaaggttttgccaaggacgcagctttctatactggccgcagtgaggtgcatagcgtaatgtccatgttgttctacactctgatcactgcgtttttgatcggcgtacaggcagaaccgtacacagatagcaatgtcccagaaggagactctgtccctgaagcccactggactaaacttcagcattcccttgacacagccctccgcagagcccgcagtgcccctactgcaccaatagctgcccgagtgacagggcagacccgcaacatcactgtagaccccagactgtttaagaaacggagactccactcaccccgtgtgctgttcagcacccagcctccacccacctcttcagacactctggatctagacttccaggcccatggtacaatccctttcaacaggactcaccggagcaagcgctcatccacccacccagtcttccacatgggggagttctcagtgtgtgacagtgtcagtgtgtgggttggagataagaccacagccacagacatcaagggcaaggaggtgacagtgctggccgaggtgaacattaacaacagtgtattcagacagtacttttttgagaccaagtgccgagcctccaatcctgttgagagtgggtgccggggcatcgactccaaacactggaactcatactgcaccacgactcacaccttcgtcaaggcgttgacaacagatgagaagcaggctgcctggaggttcatccggatagacacagcctgtgtgtgtgtgctcagcaggaaggctacaagaagaggctgacttgcctgcagcccccttccccacctgccccctccacactctcctgggcccctccctacctcagcctgtaaattattttaaattataaggactgcatgataatttatcgtttatacaattttaaagacattatttattaaattttcaaagcatcctgtataccga

SEQ ID NO: 7 - protein, 307 aa, Mus musculus
MLCLKPVKLGSLEVGHGQHGGVLACGRAVQGAGWHAGPKLTSVSGPNKGFAKDAAFYTGRSEVHSVMSMLFYTLITAFLIGVQAEPYTDSNVPEGDSVPEAHWTKLQHSLDTALRRARSAPTAPIAARVTGQTRNITVDPRLFKKRRLHSPRVLFSTQPPPTSSDTLDLDFQAHGTIPFNRTHRSKRSSTHPVFHMGEFSVCDSVSVWVGDKTTATDIKGKEVTVLAEVNINNSVFRQYFFETKCRASNPVESGCRGIDSKHWNSYCTTTHTFVKALTTDEKQAAWRFIRIDTACVCVLSRKATRRG

SEQ ID NO: 8 - DNA, 1069 bp, Mus musculus
cagcacggcagagagcgcctggagccggaggggagcgcatcgagttttggcctgtggtcgtgcagtccagggggctggatggcatgctggacccaagctcacctcagtgtctgggcccaataaaggttttgccaaggacgcagctttctatactggccgcagtgaggtgcatagcgtaatgtccatgttgttctacactctgatcactgcgtttttgatcggcgtacaggcagaaccgtacacagatagcaatgtcccagaaggagactctgtccctgaagcccactggactaaacttcagcattcccttgacacagccctccgcagagcccgcagtgcccctactgcaccaatagctgcccgagtgacagggcagacccgcaacatcactgtagaccccagactgtttaagaaacggagactccactcaccccgtgtgctgttcagcacccagcctccacccacctcttcagacactctggatctagacttccaggcccatggtacaatccctttcaacaggactcaccggagcaagcgctcatccacccacccagtcttccacatgggggagttctcagtgtgtgacagtgtcagtgtgtgggttggagataagaccacagccacagacatcaagggcaaggaggtgacagtgctggccgaggtgaacattaacaacagtgtattcagacagtacttttttgagaccaagtgccgagcctccaatcctgttgagagtgggtgccggggcatcgactccaaacactggaactcatactgcaccacgactcacaccttcgtcaaggcgttgacaacagatgagaagcaggctgcctggaggttcatccggatagacacagcctgtgtgtgtgtgctcagcaggaaggctacaagaagaggctgacttgcctgcagcccccttccccacctgccccctccacactctcctgggcccctccctacctcagcctgtaaattattttaaattataaggactgcatgataatttatcgtttatacaattttaaagacattatttattaaattttcaaagcatcctgtataccga

SEQ ID NO: 9 - protein, 241 aa, Mus musculus
MSMLFYTLITAFLIGVQAEPYTDSNVPEGDSVPEAHWTKLQHSLDTALRRARSAPTAPIAARVTGQTRNITVDPRLFKKRRLHSPRVLFSTQPPPTSSDTLDLDFQAHGTIPFNRTHRSKRSSTHPVFHMGEFSVCDSVSVWVGDKTTATDIKGKEVTVLAEVNINNSVFRQYFFETKCRASNPVESGCRGIDSKHWNSYCTTTHTFVKALTTDEKQAAWRFIRIDTACVCVLSRKATRRG

SEQ ID NO: 10 - protein, 151 aa, Homo sapiens
MGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQASSSSKAPPPSLPSPSRLPGPSDTPILPQ

SEQ ID NO: 11 - protein, 163 aa, Homo sapiens
MSKGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQASSSSKAPPPSLPSPSRLPGPSDTPILPQ

SEQ ID NO: 12 - protein, 165 aa, Homo sapiens
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 13 - protein, 28 aa, Homo sapiens
SSSSKAPPPSLPSPSRLPGPSDTPILPQ

SEQ ID NO: 14 - DNA, 37 bp, Artificial Sequence
  Description of Artificial Sequence Synthetic primer
atctcgagcaccatgtccatgttgttctacactctga

SEQ ID NO: 15 - DNA, 39 bp, Artificial Sequence
  Description of Artificial Sequence Synthetic primer
tggagccttggaagagctagaggctcttctcacagcctt

SEQ ID NO: 16 - DNA, 39 bp, Artificial Sequence
  Description of Artificial Sequence Synthetic primer
aaggctgtgagaagagcctctagctcttccaaggctcca

SEQ ID NO: 17 - DNA, 30 bp, Artificial Sequence
  Description of Artificial Sequence Synthetic primer
tttgcggccgcttactactggggcagaata

SEQ ID NO: 18 - protein, 6 aa, Artificial Sequence
  Description of Artificial Sequence Synthetic 6xHis tag
HHHHHH

SEQ ID NO: 19 - protein, 7 aa, Homo sapiens
SSSHPIF

SEQ ID NO: 20 - protein, 6 aa, Homo sapiens
YAEHKS

(19) United States
(12) Patent Application Publication
(10) Pub. No.: US 2022/0339251 A1
(43) Pub. Date: Oct. 27, 2022

(54) COMPOSITIONS AND METHODS FOR RECOMBINANT NERVE GROWTH FACTOR

(71) Applicant: Vivibaba, Inc, Culver City, CA (US)

(72) Inventor: Jing Wen, Culver City, CA (US)

(21) Appl. No.: 17/542,108

(22) Filed: Dec. 3, 2021

Related U.S. Application Data
(63) Continuation of application No. 15/892,527, filed on Feb. 9, 2018, now abandoned.
(60) Provisional application No. 62/457,499, filed on Feb. 10, 2017.

(51) Int. Cl. A61K 38/18; C12N 15/62; C12N 15/85; A61K 47/69; A61P 25/02; A61K 9/127; C07K 17/02; A61P 25/28; C07K 14/48

(52) U.S. Cl. CPC A61K 38/185; C12N 15/62; C12N 15/85; A61K 47/6911; A61P 25/02; A61K 9/127; C07K 17/02; A61P 25/28; C07K 14/48; Y02A 50/30

(57) ABSTRACT

The present application provides nerve growth factor (NGF) variants with improved in vivo stability, methods for producing and purifying NGF variants, as well as potential therapeutic applications.

### RELATED APPLICATIONS

This application claims the benefit of priority to U.S. Provisional Patent Application Ser. No. 62/457,499, filed Feb. 10, 2017, which is herein incorporated by reference in its entirety.

### BACKGROUND OF THE INVENTION

Nerve growth factor (NGF) was first isolated from mouse sarcomas by the Nobel Laureate Rita Levi-Montalcini in 1953, and was the first neurotrophic factor to be discovered. NGF primarily regulates the survival, differentiation and proliferation of sympathetic neurons and sensory nerves from the neural crest. It also plays an important role in the recovery of neurological function and neural regeneration processes.

NGF is found abundant in various sources, and the submandibular gland of mice is one of the most intensively studied sources of NGF. Other sources include snake venom, the seminal vesicles of bulls, Guinea-pig prostate and human placenta. Of these sources, the gene sequence of mouse NGF is most highly homologous (>90%) to that of the human, and therefore mouse NGF isolated from submandibular glands and human NGF from placenta have been used in clinical applications, mainly for the treatment of optic nerve injuries. Additional conditions responsive to treatment with NGF include toxic neuropathy, peripheral neuropathy, facial neuropathy, and others.

Currently, mouse NGF is administered by intramuscular injection. Because of the short half-life of mouse NGF, daily injection (30 μg mouse NGF) is required for a course of 4 weeks. Major adverse effects include local pain caused by the multiple injections over the course of therapy. Preliminary pharmacokinetic data have shown that human NGF exhibits a similar in vivo half-life to mouse NGF, and thus would likely require a similar drug administration frequency, i.e., daily injection, in clinical use. However, daily injections are extremely inconvenient and uncomfortable to patients.

Therefore, there is a need for improved therapeutic forms of human NGF (NGF) that can be administered more conveniently.

### SUMMARY OF THE INVENTION

The present invention relates, in part, to a long-lasting recombinant human nerve growth factor (rhNGF) that has a longer half-life in patients. Specifically, compared to unaltered rhNGF, the long-lasting nerve growth factor exhibits similar biological activity, but the in vivo half-life is substantially longer, as demonstrated in animal studies. The NGF variant polypeptide described herein comprises an additional polypeptide portion in addition to an NGF portion. In some embodiments, the additional polypeptide portion increases the in vivo stability (e.g., the half-life) of the NGF portion. In some embodiments, such additional polypeptide comprises a human chorionic gonadotropin (hCG) or a biologically active fragment thereof. In some preferred embodiments, such additional polypeptide comprises at least a carboxy-terminal portion (CTP) of hCG.

In one aspect, the present invention provides a polypeptide comprising

i) a first portion comprising a full-length nerve growth factor (NGF) polypeptide sequence or a biologically active fragment thereof; and

ii) a second portion comprising an additional polypeptide that increases the half-life of the NGF polypeptide sequence or biologically active fragment thereof.

In some embodiments, the first portion of the polypeptide described herein comprises a full-length NGF polypeptide sequence. In other embodiments, the first portion of the polypeptide described herein comprises a biologically active fragment of NGF.

The polypeptide described herein may have a mammal origin. For example, such polypeptide, or its first portion and/or its second portion, comprises a mammalian sequence, such as a human or a mouse sequence. In some embodiments, the first portion of the polypeptide described herein comprises a human NGF sequence. Such human NGF sequence may be at least 70%, 75%, 80%, 85%, 90%, 91%, 92%, 93%, 94%, 95%, 96%, 97%, 98%, 99%, or more identical to SEQ ID NO:5. In some embodiments, the human NGF sequence is at least 70% identical to SEQ ID NO:5. In some embodiments, the human NGF sequence is at least 80% identical to SEQ ID NO:5. In some embodiments, the human NGF sequence is at least 90% identical to SEQ ID NO:5. In some embodiments, the human NGF sequence is at least 95% identical to SEQ ID NO:5. In some embodiments, the human NGF sequence is at least 99% identical to SEQ ID NO:5. In some embodiments, the human NGF sequence is SEQ ID NO:5. In some embodiments, the human NGF sequence is encoded by a polynucleotide specifically hybridize under stringent conditions to a polynucleotide that encodes a polypeptide having a sequence complementary to SEQ ID NO:5.

In some embodiments, the first portion of the polypeptide described herein is capable of binding to at least one binding partner of NGF, optionally wherein the at least one binding partner of NGF is a tropomyosin receptor kinase A (TrkA) or a low-affinity NGF receptor (LNGFR/p75NTR). In some embodiments, the first portion of the polypeptide described herein comprises a biologically active fragment of NGF, wherein such biological activity is measured by its interaction with at least one of NGF binding partner, such as TrkA and LNGFR/p75NTR. In some embodiments, the first portion of the polypeptide described herein comprises amino acid residues from position 122 to position 241 of SEQ ID NO:5.

In some embodiments, the second portion of the polypeptide described herein comprises a full-length human chorionic gonadotropin (HCG), or a biologically active fragment thereof.

In some embodiments, the second portion of the polypeptide described herein comprises a human HCG sequence, such as one of SEQ ID Nos:10-12. In some embodiments, the second portion comprises a carboxyl-terminal portion (CTP) of HCG. In some embodiments, the second portion of the polypeptide described herein comprises at least 70%, 75%, 80%, 85%, 90%, 91%, 92%, 93%, 94%, 95%, 96%, 97%, 98%, 99%, or more identical to SEQ ID NO:13. In some embodiments, the second portion is at least 70% identical to SEQ ID NO:13. In some embodiments, the second portion is at least 75% identical to SEQ ID NO:13. In some embodiments, the second portion is at least 80% identical to SEQ ID NO:13. In some embodiments, the second portion is at least 90% identical to SEQ ID NO:13. In some embodiments, the second portion is at least 95% identical to SEQ ID NO:13. In some embodiments, the second portion is at least 99% identical to SEQ ID NO:13. In some embodiments, the second portion comprises SEQ ID NO:13. In some embodiments, the second portion is encoded by a polynucleotide that specifically hybridizes under stringent conditions to a polynucleotide that encodes a polypeptide having a sequence complementary to SEQ ID NO:13.

In some embodiments, the second portion of the polypeptide described herein comprises at least one glycosylation site.

In some embodiments, the polypeptide described herein is a fusion protein. For example, the first portion and the second portion of the polypeptide may be fused together, with or without a linker (e.g., a peptide linker). The first portion may be fused to the N-terminus of the second portion, or to the C-terminus of the second portion. Multiple copies of the first portion and/or the second portion may be fused together.

In some embodiments, the polypeptide described herein comprises an amino acid sequence at least 70%, 75%, 80%, 85%, 90%, 91%, 92%, 93%, 94%, 95%, 96%, 97%, 98%, 99%, or more identical to SEQ ID NO:2 or 3. In some embodiments, the polypeptide sequence is at least 70% identical to SEQ ID NO:2 or 3. In some embodiments, the polypeptide sequence is at least 75% identical to SEQ ID NO: 2 or 3. In some embodiments, the polypeptide sequence is at least 80% identical to SEQ ID NO:2 or 3. In some embodiments, the polypeptide sequence is at least 90% identical to SEQ ID NO:2 or 3. In some embodiments, the polypeptide sequence is at least 95% identical to SEQ ID NO:2 or 3. In some embodiments, the polypeptide sequence is at least 99% identical to SEQ ID NO:2 or 3. In some embodiments, the polypeptide sequence comprise SEQ ID NO:2 or 3. In some embodiments, the polypeptide is encoded by a polynucleotide that specifically hybridizes under stringent conditions to a polynucleotide that encodes a polypeptide having a sequence complementary to SEQ ID NO:2 or 3.

In some embodiments, the polypeptide described herein exhibits an increased half-life in vivo relative to its first portion. For example, the polypeptide may have an in vivo half-life at least 1.5, 1.6, 1.7, 1.8, 1.9, 2.0, 2.1, 2.2, 2.3, 2.4, 2.5, 2.6, 2.7, 2.8, 2.9, 3.0, or more times the in vivo half-life of its first portion (i.e., the NGF polypeptide sequence) alone, e.g., in a human. In some embodiments, the polypeptide has in vivo half-life at least 2.5 times the in vivo half-life of the NGF polypeptide sequence alone.

In some embodiments, the polypeptide described herein, or its first or second portion, further comprises a label, such as a purification label and/or tag (e.g., GST, FLAG, hexa-his (His6), etc.) and/or a fluorescent tag.

In another aspect, the present invention provides a polynucleotide encoding a polypeptide described herein. For example, such polynucleotide may comprise a nucleic acid sequence at least 70%, 75%, 80%, 85%, 90%, 91%, 92%, 93%, 94%, 95%, 96%, 97%, 98%, 99%, or more identical to SEQ ID NO:1. In some embodiments, the polynucleotide sequence is at least 70% identical to SEQ ID NO:1. In some embodiments, the polynucleotide sequence is at least 80% identical to SEQ ID NO:1. In some embodiments, the polynucleotide sequence is at least 90% identical to SEQ ID NO:1. In some embodiments, the polynucleotide sequence is at least 95% identical to SEQ ID NO:1. In some embodiments, the polynucleotide sequence is at least 99% identical to SEQ ID NO:1. In some embodiments, the polynucleotide sequence comprises SEQ ID NO:1. In some embodiments, the polynucleotide sequence specifically hybridizes under stringent conditions to a sequence complementary to SEQ ID NO:1. In some embodiments, the polynucleotide described herein further comprises a detection label, such as a purification label and/or tag (e.g., GST, FLAG, hexa-his (His6), etc.) and/or a fluorescent tag. In some embodiments, the stringent conditions comprise hybridization in 50% v/v formamide, 5×SSC, 2% w/v blocking agent, 0.1% N-lauroylsarcosine, 0.3% SDS at 65° C. overnight and washing in 5×SSC at about 65° C.

In another aspect, the present invention provides an expression vector capable of expressing a polypeptide and/or polynucleotide described herein. Such expression vector may be a plasmid, a cosmid, a viral vector, etc., with our without genetic modifications.

In another aspect, the present invention provides a host cell comprising the expression vector, the polypeptide, or the polynucleotide described herein. Such host cell may be a bacterial cell, a yeast cell, an insect cell, a chicken cell, or a mammalian cell (such as CHO, Hela, HT293, or other cells). In some embodiments, the host cell described herein is immortalized.

In another aspect, the present invention provides a method comprising

i) culturing the host cell described herein in a cell culture medium; and

ii) expressing the polypeptide described herein.

In some embodiments, the method further comprises

iii) purifying the polypeptide described herein from the cell culture medium. Through these exemplary methods, the polypeptide described herein may be produced, separated, and eventually purified for further uses.

In another aspect, the present invention also provides a composition comprising a polypeptide, polynucleotide, expression vector, and/or host cell described herein. In certain such embodiments, the composition is a pharmaceutical composition that further comprises a pharmaceutically acceptable carrier.

In another aspect, the present invention provides a method of treating a disease or disorder related to deficient and/or defective nerve growth factor (NGF), comprising administering to the subject a polypeptide, composition, or pharmaceutical composition, described herein. Such disease or disorder may be any one of neuronal disorders described herein, such as neuronal degeneration. In some embodiments, the method further comprises administering to the subject an additional agent and/or therapy to treat the disease or disorder.

In another aspect, the present invention provides a method of promoting growth and/or proliferation of neurons, comprising administering to the subject a polypeptide, composition, or pharmaceutical composition described herein.

In some embodiments, the subject is a mammal, such as a non-human mammal (e.g., a mouse, a dog, a cat, etc.) or a human. In preferred embodiments, the subject is a human.

### BRIEF DESCRIPTION OF THE DRAWINGS

FIG. 1 depicts a scheme of the structure of the pCI-neo/NGF-CTP plasmid.

FIG. 2 depicts a gel electrophoresis of the purified rhNGF-CTP protein.

FIG. 3 shows the rat plasma concentration profile of rhNGF and rhNGF-CTP in a pharmacokinetics study after i.v. injection.

### DETAILED DESCRIPTION OF THE INVENTION

The present invention relates, in part, to a discovery of nerve growth factor (NGF) variants, especially NGF variants comprising a full-length NGF sequence or a biologically active fragment thereof, and another polypeptide. Such NGF variants may be more stable in vitro, ex vivo, and/or in vivo than wild-type NFG proteins (e.g., with a longer half-lives).

Accordingly, polypeptides, polynucleotides, and compositions of NGF variants are provided that maintain at least part or all of wild-type NGF activity. In some embodiments, the NGF variants comprise a full-length NGF sequence, or a biologically active fragment thereof. In some embodiments, the NGF variants comprise another polypeptide, preferably a heterologous polypeptide, e.g., to form a fusion protein. For example, the NGF portion and the heterologous polypeptide portion of an NGF variant described herein may be fused together with or without a linker. In some embodiments, the NGF portion is fused to the N-terminus of the heterologous polypeptide. In other embodiments, the NGF portion is fused to the C-terminus of the heterologous polypeptide. The amino acid sequence of the NGF portion of the NGF variants described herein may be derived from the wild-type NGF sequences, or by the substitution, insertion or deletion of one or more amino acids of a parent NGF amino acid sequence, such as a wild-type NGF sequence. An NGF variant may retain at least 70% amino acid sequence identity with the wild-type NGF molecule or the parent NGF molecule from which it is derived. Useful quantities of these NGF variants can be prepared using recombinant DNA techniques.

Another aspect of the present invention provides recombinant nucleic acids encoding the NGF variants, and expression vectors and host cells containing these nucleic acids.

Another aspect of the present invention provides methods for producing the NGF variants, such as methods using nucleic acids, vectors and host cells of the invention. In some embodiments, a host cell transformed with an expression vector containing a nucleic acid encoding an NGF variant is cultured to allow expression of the nucleic acid to produce a recombinant NGF variant.

Furthermore, methods and compositions for treating a neurodegenerative disease or disorder (e.g., neuronal disorders, such as neuronal degeneration) in a subject are provided, use the NGF variants and related therapeutics described herein.

Other advantages and aspects of the invention will become apparent from the following detailed description, the figures, and the claims.

### I. Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent (such as the polypeptides and/or compositions described herein) to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo. In some embodiments, the agent is orally administered. In other embodiments, the agent is administered through an injection route described herein.

The term “increased/decreased amount” or “increased/decreased level” refers to increased or decreased absolute and/or relative amount and/or value of a molecule (e.g., NGF) in a subject, as compared to the amount and/or value of the same molecule in the same subject in a prior time and/or in a normal and/or control subject.

The amount of a molecule (e.g., NGF) in a subject is “significantly” higher or lower than the normal amount of the molecule, if the amount of the molecule is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the molecule in the subject can be considered “significantly” higher or lower than the normal amount if the amount is at least about two, and preferably at least about three, four, or five times, higher or lower, respectively, than the normal amount of the molecule. Such “significance” can also be applied to any other measured parameter described herein, such as for expression, inhibition, cytotoxicity, cell growth, and the like.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. In some embodiments, the host cell described herein is a mammalian cell (such as Chinese Hamster Ovary (CHO) cells, Hela cells, etc.). In some embodiments, the host cell is immortalized.

As used herein, the term “interaction,” when referring to an interaction between two molecules (e.g., NGF variants and their binding partners), refers to the physical contact (e.g., binding) of the molecules with one another. Generally, such an interaction results in an activity (which produces a biological effect) of one or both of said molecules. The activity may be a direct activity of one or both of the molecules. Alternatively, one or both molecules in the interaction may be prevented from binding their ligand, and thus be held inactive with respect to ligand binding activity (e.g., binding its ligand and triggering or inhibiting an immune response). To inhibit such an interaction results in the disruption of the activity of one or more molecules involved in the interaction. To enhance such an interaction is to prolong or increase the likelihood of said physical contact, and prolong or increase the likelihood of said activity.

As used herein, an “isolated protein” refers to a protein (e.g., the NGF variant polypeptides) that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations, in which compositions of the invention are separated from cellular components of the cells from which they are isolated or recombinantly produced. In one embodiment, the language “substantially free of cellular material” includes preparations of having less than about 30%, 20%, 10%, or 5% (by dry weight) of cellular material. When an antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

In some embodiments, a therapeutic composition comprising the NGF variant polypeptide described herein is used for treating a disease or disorder in a subject. Such disease or disorder may include, e.g., any disease related to a deficiency in the amount, level, and/or activity of endogenous NGF gene, mRNA, and/or protein, such as a neuronal disorder (e.g., neuronal degeneration), etc. In some embodiments, the therapeutic composition described herein further comprises another agent capable of treating the disease or disorder described herein.

In some embodiments, the subject described herein has a neuronal disorder (such as neuronal degeneration). NGF variants described herein are believed to be useful in promoting the development, maintenance, or regeneration of neurons in vitro and in vivo, including central (brain and spinal cord), peripheral (sympathetic, parasympathetic, sensory, and enteric neurons), and motor neurons. Accordingly, NGF variants described herein are useful in methods for treating a variety of neurologic diseases and disorders. In preferred embodiments, the formulations of the present invention are administered to a patient to treat one or more neural disorders or conditions. By “neural disorders” herein is meant disorders of the central and/or peripheral nervous system that are associated with neuron degeneration or damage. Specific examples of neural disorders include, but are not limited to, Alzheimer's disease, Parkinson's disease, Huntington's chorea, stroke, ALS, peripheral neuropathies, and other conditions characterized by necrosis or loss of neurons, whether central, peripheral, or motor neurons, in addition to treating damaged nerves due to trauma, burns, kidney dysfunction, injury, and the toxic effects of chemotherapeutics used to treat cancer and AIDS. For example, peripheral neuropathies associated with certain conditions, such as neuropathies associated with diabetes, AIDS, or chemotherapy may be treated using the formulations of the present invention. The compounds and compositions also may be useful in culture media for culturing nerve cells in vitro or ex vivo.

In various embodiments of the invention, an NGF variant may be administered to patients whose nervous system has been damaged by trauma, surgery, stroke, ischemia, infection, metabolic disease, nutritional deficiency, malignancy, or toxic agents, to promote the survival or growth of neurons, or in any condition are treatable with NGF, NT-3, BDNF or NT4-5. Without limitation, the treatment or effect vary with the particular trk-binding function or functions present in the NGF variant. For example, NGF variants described herein can be used to promote the survival or growth of motor neurons that are damaged by trauma or surgery. Also, NGF variants described herein can be used to treat motor neuron disorders, such as amyotrophic lateral sclerosis (Lou Gehrig's disease), Bell's palsy, and various conditions involving spinal muscular atrophy, or paralysis. NGF variants described herein can be used to treat human neurodegenerative disorders, such as Alzheimer's disease, Parkinson's disease, epilepsy, multiple sclerosis, Huntington's chorea, Down's Syndrome, nerve deafness, and Meniere's disease.

NGF variants described herein can be used as cognitive enhancers, e.g., to enhance learning, including in patients with dementia or trauma. Alzheimer's disease, which has been identified by the National Institutes of Aging as accounting for more than 50% of dementia in the elderly, is also the fourth or fifth leading cause of death in Americans over 65 years of age. Four million Americans, 40% of Americans over age 85 (the fastest growing segment of the U.S. population), have Alzheimer's disease. Twenty-five percent of all patients with Parkinson's disease also suffer from Alzheimer's disease-like dementia. And in about 15% of patients with dementia, Alzheimer's disease and multi-infarct dementia coexist. The third most common cause of dementia, after Alzheimer's disease and vascular dementia, is cognitive impairment due to organic brain disease related directly to alcoholism, which occurs in about 10% of alcoholics. However, the most consistent abnormality for Alzheimer's disease, as well as for vascular dementia and cognitive impairment due to organic brain disease related to alcoholism, is the degeneration of the cholinergic system arising from the basal forebrain (BF) to both the codex and hippocampus (Bigl et al. in Brain Cholinergic Systems, M. Steriade and D. Biesold, eds., Oxford University Press, Oxford, pp. 364-386 (1990)). And there are a number of other neurotransmitter systems affected by Alzheimer's disease (Davies Med. Res. Rev. 3:221 (1983)). However, cognitive impairment, related for example to degeneration of the cholinergic neurotransmitter system, is not limited to individuals suffering from dementia. It has also been seen in otherwise healthy aged adults and rats. Studies that compare the degree of learning impairment with the degree of reduced cortical cerebral blood flow in aged rats show a good correlation (Berman et al. Neurobiol. Aging 9:691 (1988)). In chronic alcoholism the resultant organic brain disease, like Alzheimer's disease and normal aging, is also characterized by diffuse reductions in cortical cerebral blood flow in those brain regions where cholinergic neurons arise (basal forebrain) and to which they project (cerebral cortex) (Lofti et al., Cerebrovasc. and Brain Metab. Rev 1:2 (1989)). Such dementias can be treated by administration of NGF variants described herein.

Further, NGF variants described herein may be used to treat neuropathy, and especially peripheral neuropathy. “Peripheral neuropathy” refers to a disorder affecting the peripheral nervous system, most often manifested as one or a combination of motor, sensory, sensorimotor, or autonomic neural dysfunction. The wide variety of morphologies exhibited by peripheral neuropathies can each be attributed uniquely to an equally wide number of causes. For example, peripheral neuropathies can be genetically acquired, can result from a systemic disease, or can be induced by a toxic agent. Examples include but are not limited to diabetic peripheral neuropathy, distal sensorimotor neuropathy, or autonomic neuropathies such as reduced motility of the gastrointestinal tract or atony of the urinary bladder. Examples of neuropathies associated with systemic disease include post-polio syndrome or AIDS-associated neuropathy; examples of hereditary neuropathies include Charcot-Marie-Tooth disease, Refsum's disease, Abetalipoproteinemia, Tangier disease, Krabbe's disease, Metachromatic leukodystrophy, Fabry's disease, and Dejerine-Sottas syndrome; and examples of neuropathies caused by a toxic agent include those caused by treatment with a chemotherapeutic agent such as vincristine, cisplatin, methotrexate, or 3′-azido-3′-deoxythymidine.

Accordingly, a method of treating a neural disorder in a mammal comprising administering to the mammal an NGF variant described herein is provided. Preferably, the neural disorder is a peripheral neuropathy, more preferably diabetic peripheral neuropathy, chemotherapy-induced peripheral neuropathy, or HIV-associated neuropathy. Preferably the peripheral neuropathy affects motor neurons.

As used herein, the term “nerve growth factor” or “NGF” refers to a group of neurotrophic factors and neuropeptides primarily involved in the regulation of growth, maintenance, proliferation, and survival of certain target neurons, especially those that transmit pain, temperature, and touch sensations (sensory neurons). The term “NGF” described herein include the wild-type and any mutated, substituted, and/or modified beta chain of NGF (NGFβ) from any species described herein, including those exemplified in Table 1, unless specified otherwise. It is perhaps the prototypical growth factor, in that it was one of the first to be described. Since it was first isolated by Nobel Laureates Rita Levi-Montalcini and Stanley Cohen in 1956, numerous biological processes involving NGF have been identified, two of them being the survival of pancreatic beta cells and the regulation of the immune system. NGF is initially in a 7S, 130-kDa complex of 3 proteins—Alpha-NGF, Beta-NGF, and Gamma-NGF (2:1:2 ratio) when expressed. This form of NGF is also referred to as proNGF (NGF precursor). The gamma subunit of this complex acts as a serine protease, and cleaves the N-terminal of the beta subunit, thereby activating the protein into functional NGF. Human NGF beta subunit contains 241 amino acids of about 26959 Da (Gene Bank ID: NP_002497.2), encoded by a nucleic acid sequence as set forth in Gene Bank ID: NM_002506.2. Its domain structure is also well known under UniProt ID no. P01138. Specifically, for the human NGF sequence as shown in SEQ ID NO: 5, amino acid residues from position 1 to position 18 represent a signal peptide, followed by a propeptide region from position 19 to position 121 and a NGF mature polypeptide ranging from position 122 to position 241 (SEQ ID NO:6). Known amino acid modifications include N-linked glycosylation sites on positions 69 and 114 and disulfide bonds between positions 136 and 201, 179 and 229, and 189 and 231 of SEQ ID NO:5. NGFβ homologs in other organisms are also well known in the art, including the Chimpanzee NGFβ (NM_001012437.1 and NP_001012439.1), the Rhesus monkey NGFβ (XM_015148902.1 and XP_015004388.1, and) 34_015148898.1 and XP_015004384.1), the dog NGFβ (NM_001194950.1 and NP_001181879.1), the cattle NGFβ (NM_001099362.1 and NP_001092832.1), the mouse NGFβ (NM_001112698.2 and NP_001106168.1, and NM_013609.3 and NP_038637.1), the rat NGFβ (NM_001277055.1 and NP_001263984.1), the chicken NGFβ (NM_001293108.1 and NP_001280037.1, and NM_001293109.1 and NP_001280038.1), and the tropical clawed frog NGFβ (NM_001129924.1 and NP_001123396.1). For mouse NGFβ, NM_013609.3 refers to the longer transcript variant 1 and encodes the longer isoform A (NP_038637.1), while NM_001112698.2 refers to variant 2 which contains a distinct 5′ UTR and lacks an in-frame portion of the 5′ coding region compared to variant 1. The resulting isoform B (NP_001106168.1) has a shorter N-terminus compared to isoform A. NGF binds to at least a tropomyosin receptor kinase A (TrkA) and a low-affinity NGF receptor (LNGFR/p75NTR). The NGF portion described herein may comprise any fragment of full-length NGF. In some embodiments, the NGF portion comprises at least the domains for NGF interaction with TrkA or LNGFR. For example, the NGF portion may comprise the amino acid residues from position 122 to position 241 of SEQ ID NO:5. The NGF portion described herein may comprise NGF sequences comprising substitutions, mutations, modifications, and/or deletions. Some exemplary NGF sequences are listed in Table 1. For example, NGF sequences as described in U.S. Pat. Nos. 6,333,310 and 7,452,863 are also included in the scope of the present invention.

The NGF variant polypeptide described herein comprises an NGF portion, which comprises a full-length NGF polypeptide (either a NGF precursor polypeptide or a mature NGF polypeptide) or a biologically active fragment thereof. The term “biologically active fragment” refers to a portion of a wild-type NGF polypeptide of any species, comprising any possible substitutions, mutations, deletions, insertions, fusions, and/or other modification methods, while maintaining at least one of biological functions of the wild-type NGF polypeptide. The term “biological function” of NGF refers to, generally, its ability to promote growth, maintenance, and survival of neurons and axons, including facilitating myelin sheath repair and other related functions. Specifically, the term “biological function” of NGF may also refer to its signaling function through its binding to TrkA and/or p75NTR. Multiple tests for these general and specific functions are known in the art.

NGF has a number of domains which can affect NGF specificity when modified. The N-terminal amino acids of NGF are the main region in NGF responsible for trkA binding. Significant losses of biological activity and receptor binding were observed with purified homodimers of human and mouse NGF, representing homogenous truncated forms modified at the amino and carboxy termini. A truncated mature NGF species comprising 109 amino acids, (10-118)hNGF, resulting from the loss of the first 9 residues of the N-terminus and the last two residues from the C-terminus of purified recombinant human mature NGF, is 300-fold less efficient in displacing mouse ¹²⁵I-NGF from the human trkA receptor compared to (1-118)hNGF. It is 50- to 100-fold less active in dorsal root ganglion and sympathetic ganglion survival compared to (1-118)hNGF. A modification of the 10-amino-acid-N-terminal region may result in a reduction or elimination of TrkA binding. For example, the U.S. Pat. No. 6,333,310 describes a deletion or substitution of the 7-terminal amino acids (SSSHPIF) of NGF with the N-terminal amino acids of NT-3 (YAEHKS), resulting in an NGF variant with reduced or absent trkA-binding activity. In addition, PCT Publication no. WO 95/33829 discloses NGF variants that lack NGF activity. In the present disclosure, the NGF portion of the NGF variant polypeptides may comprise a NGF polypeptide having intact, increased, or decreased binding ability to Trk A and/or p75NTR. In some embodiments, the NGF polypeptide has a binding affinity to Trk A and/or p75NTR at least as same as the wild type NGF. In some embodiments, the NGF polypeptide has a higher binding affinity to Trk A and/or p75NTR relative to wild type NGF. In some other embodiments, the NGF polypeptide has a weaker binding affinity to Trk A and/or p75NTR relative to wild type NGF. In some embodiments, a trkB-recruiting modification may be combined with a trkA-reducing modification to yield a variant that binds both trkC and trkB, but not trkA.

The NGF variant polypeptide described herein comprises an additional polypeptide in addition to an NGF portion. In some embodiments, the additional polypeptide increases the in vivo stability (e.g., the half-life) of the NGF portion. Such additional polypeptide may be any polypeptide with such function. For example, such additional polypeptide may be an immunoglobulin or a biologically active fragment thereof. In some embodiments, such additional polypeptide comprises an Fc (Fragment, crystallizable) region of any type of IgG. In some embodiments, such additional polypeptide comprises a human chorionic gonadotropin (hCG) or a biologically active fragment thereof. In some preferred embodiments, such additional polypeptide comprises at least a carboxyl-terminal portion (CTP) of hCG.

As used herein, the term “human chorionic gonadotropin” or “hCG” refers to a group of hormone produced by the placenta after implantation. The presence of hCG is detected in some pregnancy tests (HCG pregnancy strip tests). Some cancerous tumors produce this hormone; therefore, elevated levels measured when the patient is not pregnant can lead to a cancer diagnosis and, if high enough, paraneoplastic syndromes. However, it is not known whether this production is a contributing cause or an effect of carcinogenesis. The pituitary analog of hCG, known as luteinizing hormone (LH), is produced in the pituitary gland of males and females of all ages. Regarding endogenous forms of hCG, there are various ways to categorize and measure them, including total hCG, C-terminal peptide total hCG, intact hCG, free β-subunit hCG, β-core fragment hCG, hyperglycosylated hCG, nicked hCG, alpha hCG, and pituitary hCG. Regarding pharmaceutical preparations of hCG from animal or synthetic sources, there are many gonadotropin preparations, some of which are medically justified and others of which are of a quack nature. As of Dec. 6, 2011, the United States Food and Drug Administration has prohibited the sale of “homeopathic” and over-the-counter hCG diet products and declared them fraudulent and illegal. Human chorionic gonadotropin is a glycoprotein composed of 237 amino acids with a molecular mass of 25.7 kDa. It is heterodimeric, with an a (alpha) subunit identical to that of luteinizing hormone (LH), follicle-stimulating hormone (FSH), thyroid-stimulating hormone (TSH), and β (beta) subunit that is unique to hCG. The α (alpha) subunit is 92 amino acids long. The β-subunit of a hCG gonadotropin isoform (beta-hCG) contains 145 amino acids, encoded by six highly homologous genes that are arranged in tandem and inverted pairs on chromosome 19q13.3. The two subunits create a small hydrophobic core surrounded by a high surface area-to-volume ratio: 2.8 times that of a sphere. The vast majority of the outer amino acids are hydrophilic. The 3-D structure of hCG was taught by Wu et al. (1994) Structure 2:545-558. Some exemplary amino acid sequences of beta subunit of hCG are listed as SEQ ID Nos: 10-12 in Table 1. See Bahl et al. (1972) Biochem. Biophys. Res. Commun. 48:416-422 for a report of both alpha and beta chain sequences of hCG. Human chorionic gonadotropin interacts with the LHCG receptor of the ovary and promotes the maintenance of the corpus luteum during the beginning of pregnancy. This allows the corpus luteum to secrete the hormone progesterone during the first trimester. Progesterone enriches the uterus with a thick lining of blood vessels and capillaries so that it can sustain the growing fetus. The detection of hCG can be done by any methods, such as using a monoclonal antibody which, e.g., specifically binds to the β subunit of hCG, capable of differentiating hCG from LH and FSH. Such antibodies are well known in the art, including those antibodies (e.g., TA313616) from OriGene (Rockville, Md.).

A carboxy-terminal portion (CTP) of hCG can be fused to the therapeutic protein for a prolonged half-life (Fares et al. (2010) Endocrinology 151:4410-4417). CTP refers to a glycosylated amino acid sequence (28-mer, SEQ ID NO:13), derived from the human chorionic gonadotropin (HCG). The high biocompatibility, low immunogenicity of CTP, as well as its ability to significantly prolong the half-life of therapeutic proteins has been well researched. For example, Furuhashi et al. (1995 Mol Endocrinol. 9:54-63) teach that the hCG beta-subunit contains a carboxy-terminal extension bearing four serine-linked oligosaccharides (i.e., carboxy-terminal peptide (CTP)), which is important for maintaining its longer half-life compared with the other glycoprotein hormones. In fact, the entire signal for O-glycosylation is primarily contained within the CTP sequence and is not dependent on the flanking regions of the recipient protein. Represented by ELONVA®, a CTP fused follicle-stimulating hormone (FSH) developed by Merck and approved by the European Commission in 2010, is used clinically to help achieve pregnancy in women infertility treatment. ELONVA® is such designed that a single injection can replace a whole week of daily FSH injections for the patients. The hCG polypeptide and/or CTP portion described herein may comprise any hCG/CTP variants comprising substitutions, mutations, modifications, and/or deletions. Some exemplary variants are listed in Table 1 (underlined). For example, CTP variants as described in U.S. Pat. No. 6,225,449 are also included in the scope of the present invention.

The NGF variant polypeptide described herein may further comprises a third portion besides the two described herein. Such third portion may comprise a fusion domain capable of improving the function and/or stability of the NGF portion. Well known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the ALK1 ECD polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain preferred embodiments, an NGF variant polypeptide is fused with a domain that stabilizes the NGF polypeptide in vivo (a “stabilizer” domain). By “stabilizing” is meant anything that increases serum half-life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of fusion domains that may be selected include multimerizing (e.g., dimerizing, tetramerizing) domains and functional domains. In addition, a localization sequence may be added to help the NGF variant to localize to a specific cell, tissue, or organ. For example, multiple sequences are known in the art to localize a protein to the nervous system or other tissues. By such sequences, the recombinant NGF variants may be specifically delivered to a targeted cell, tissue, or organ for better function and less potential side effects.

Different portions of the NGF variants described herein may be fused together as a fusion protein, with or without a linker. Such linker may be any of natural or chemical linkers. For example, the poly-Gly and Gly-rich linkers taught by Priyanka et al. (2013) Protein Sci. 22:153-167.

An important and well known feature of the genetic code is its redundancy, whereby, for most of the amino acids used to make proteins, more than one coding nucleotide triplet may be employed. Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA coding for a fusion protein or polypeptide of the invention (or any portion thereof) can be used to derive the fusion protein or polypeptide amino acid sequence, using the genetic code to translate the DNA or RNA into an amino acid sequence. Likewise, for a fusion protein or polypeptide amino acid sequence, corresponding nucleotide sequences that can encode the fusion protein or polypeptide can be deduced from the genetic code (which, because of its redundancy, will produce multiple nucleic acid sequences for any given amino acid sequence). Thus, description and/or disclosure herein of a nucleotide sequence which encodes a fusion protein or polypeptide should be considered to also include description and/or disclosure of the amino acid sequence encoded by the nucleotide sequence. Similarly, description and/or disclosure of a fusion protein or polypeptide amino acid sequence herein should be considered to also include description and/or disclosure of all possible nucleotide sequences that can encode the amino acid sequence.

Finally, nucleic acid and amino acid sequence information for the loci and biomarkers of the present invention (e.g., biomarkers listed in Table 2 and the Examples) are well known in the art and readily available on publicly available databases, such as the National Center for Biotechnology Information (NCBI). For example, exemplary nucleic acid and amino acid sequences derived from publicly available sequence databases are provided below.

TABLE 1
Exemplary NGF Nucleic Acid and Amino
Acid Sequences

```
SEQ ID NO: 1
Human NGF-CTP DNA sequence
atgtccatgttgttctacactctgatcacagcttt
tctgatcggcatacaggcggaaccacactcagaga
gcaatgtccctgcaggacacaccatcccccaagcc
cactggactaaacttcagcattcccttgacactgc
ccttcgcagagcccgcagcgccccggcagcggcga
tagctgcacgcgtggcggggcagacccgcaacatt
actgtggaccccaggctgtttaaaaagcggcgact
ccgttcaccccgtgtgctgtttagcacccagcctc
cccgtgaagctgcagacactcaggatctggacttc
gaggtcggtggtgctgcccccttcaacaggactca
caggagcaagcggtcatcatcccatcccatcttcc
acaggggcgaattctcggtgtgtgacagtgtcagc
gtgtgggttggggataagaccaccgccacagacat
caagggcaaggaggtgatggtgttgggagaggtga
acattaacaacagtgtattcaaacagtactttttt
gagaccaagtgccgggacccaaatcccgttgacag
cgggtgccggggcattgactcaaagcactggaact
catattgtaccacgactcacacctttgtcaaggcg
ctgaccatggatggcaagcaggctgcctggcggtt
tatccggatagatacggcctgtgtgtgtgtgctca
gcaggaaggctgtgagaagagcctctagctcttcc
aaggctccacccccctcactcccatctcctagtag
gctccccggaccatccgacacgcctattctgcccc
agtag
SEQ ID NO: 2
Human NGF-CTP amino acid sequence
(the underlined sequence represents
the CTP fraction from hCG)
MSMLFYTLITAFLIGIQAEPHSESNVPAGHTIPQA
HWTKLQHSLDTALRRARSAPAAAIAARVAGQTRNI
TVDPRLFKKRRLRSPRVLFSTQPPREAADTQDLDF
EVGGAAPFNRTHRSKRSSSHPIFHRGEFSVCDSVS
VWVGDKTTATDIKGKEVMVLGEVNINNSVFKQYFF
ETKCRDPNPVDSGCRGIDSKHWNSYCTTTHTFVKA
LTMDGKQAAWRFIRIDTACVCVLSRKAVRRASSSS
KAPPPSLPSPSRLPGPSDTPILPQ
SEQ ID NO: 3
Mature NGF-CTP amino acid sequence
(the underlined portion represents
the CTP fraction)
SSSHPIFHRGEFSVCDSVSVWVGDKTTATDIKGKE
VMVLGEVNINNSVFKQYFFETKCRDPNPVDSGCRG
IDSKHWNSYCTTTHTFVKALTMDGKQAAWRFIRID
TACVCVLSRKAVRRASSSSKAPPPSLPSPSRLPGP
SDTPILPQ
SEQ ID NO: 4
Human NGFB cDNA Sequence (NM_002506.2,
CDS from position 170 to 895)
      1 agagagcgct gggagccgga ggggagcgca
          gcgagttttg gccagtggtc gtgcagtcca
    61 aggggctgga tggcatgctg gacccaagct
          cagctcagcg tccggaccca ataacagttt
  121 taccaaggga gcagctttct atcctggcca
          cactgaggtg catagcgtaa tgtccatgtt
  181 gttctacact ctgatcacag cttttctgat
          cggcatacag gcggaaccac actcagagag
  241 caatgtccct gcaggacaca ccatccccca
          agcccactgg actaaacttc agcattccct
  301 tgacactgcc cttcgcagag cccgcagcgc
          cccggcagcg gcgatagctg cacgcgtggc
  361 ggggcagacc cgcaacatta ctgtggaccc
          caggctgttt aaaaagcggc gactccgttc
  421 accccgtgtg ctgtttagca cccagcctcc
          ccgtgaagct gcagacactc aggatctgga
  481 cttcgaggtc ggtggtgctg cccccttcaa
          caggactcac aggagcaagc ggtcatcatc
  541 ccatcccatc ttccacaggg gcgaattctc
          ggtgtgtgac agtgtcagcg tgtgggttgg
  601 ggataagacc accgccacag acatcaaggg
          caaggaggtg atggtgttgg gagaggtgaa
  661 cattaacaac agtgtattca aacagtactt
          ttttgagacc aagtgccggg acccaaatcc
  721 cgttgacagc gggtgccggg gcattgactc
          aaagcactgg aactcatatt gtaccacgac
  781 tcacaccttt gtcaaggcgc tgaccatgga
          tggcaagcag gctgcctggc ggtttatccg
  841 gatagatacg gcctgtgtgt gtgtgctcag
          caggaaggct gtgagaagag cctgacctgc
  901 cgacacgctc cctccccctg ccccttctac
          actctcctgg gcccctccct acctcaacct
  961 gtaaattatt ttaaattata aggactgcat
          ggtaatttat agtttataca gttttaaaga
1021 atcattattt attaaatttt tggaagcata
          aa
SEQ ID NO: 5
Human NGFB Amino Acid Sequence
(NP_002497.2)
      1 msmlfytlit afligiqaep hsesnvpagh
          tipqahwtkl qhsldtalrr arsapaaaia
    61 arvagqtrni tvdprlfkkr rlrsprvlfs
          tqppreaadt qdldfevgga apfhrthrsk
  121 rssshpifhr gefsvcdsvs vwvgdkttat
          dikgkevmvl gevninnsvf kqyffetkcr
  181 dpnpvdsgcr gidskhwnsy cttthtfvka
          itmdgkqaaw rfiridtacv cvlsrkavrr
241 a
SEQ ID NO: 6
Mouse NGFB cDNA Sequence Variant 1
(NM_013609.3. CDS from position
108 to 1031)
      1 cagcacggca gagagcgcct ggagccggag
          gggagcgcat cgagtgactt tggagctggc
    61 cttatatttg gatctcccgg gcagcttttt
          ggaaactcct agtgaacatg ctgtgcctca
  121 agccagtgaa attaggctcc ctggaggtgg
          gacacgggca gcatggtgga gttttggcct
  181 gtggtcgtgc agtccagggg gctggatggc
          atgctggacc caagctcacc tcagtgtctg
  241 ggcccaataa aggttttgcc aaggacgcag
          ctttctatac tggccgcagt gaggtgcata
  301 gcgtaatgtc catgttgttc tacactctga
          tcactgcgtt tttgatcggc gtacaggcag
  361 aaccgtacac agatagcaat gtcccagaag
          gagactctgt ccctgaagcc cactggacta
  421 aacttcagca ttcccttgac acagccctcc
          gcagagcccg cagtgcccct actgcaccaa
  481 tagctgcccg agtgacaggg cagacccgca
          acatcactgt agaccccaga ctgtttaaga
  541 aacggagact ccactcaccc cgtgtgctgt
          tcagcaccca gcctccaccc acctcttcag
  601 acactctgga tctagacttc caggcccatg
          gtacaatccc tttcaacagg actcaccgga
  661 gcaagcgctc atccacccac ccagtcttcc
          acatggggga gttctcagtg tgtgacagtg
  721 tcagtgtgtg ggttggagat aagaccacag
          ccacagacat caagggcaag gaggtgacag
  781 tgctggccga ggtgaacatt aacaacagtg
          tattcagaca gtactttttt gagaccaagt
  841 gccgagcctc caatcctgtt gagagtgggt
          gccggggcat cgactccaaa cactggaact
  901 catactgcac cacgactcac accttcgtca
          aggcgttgac aacagatgag aagcaggctg
  961 cctggaggtt catccggata gacacagcct
          gtgtgtgtgt gctcagcagg aaggctacaa
1021 gaagaggctg acttgcctgc agcccccttc
          cccacctgcc ccctccacac tctcctgggc
1081 ccctccctac ctcagcctgt aaattatttt
          aaattataag gactgcatga taatttatcg
1141 tttatacaat tttaaagaca ttatttatta
          aattttcaaa gcatcctgta taccga
SEQ ID NO: 7
Mouse NGFβ Amino Acid Sequence
Isoform A (NP 038637.1)
      1 mlclkpvklg slevghgqhg gvlacgravq
          gagwhagpkl tsvsgpnkgf akdaafytgr
    61 sevhsvmsml fytlitafli gvqaepytds
          nvpegdsvpe ahwtklqhsl dtalrrarsa
  121 ptapiaarvt gqtrnitvdp rlfkkrrlhs
          prvlfstqpp ptssdtldld fqahgtipfn
  181 rthrskrsst hpvfhmgefs vcdsvsvwvg
          dkttatdikg kevtvlaevn innsvfrqyf
  241 fetkcrasnp vesgcrgids khwnsycttt
          htfvkalttd ekqaawrfir idtacvcvls
  301 rkatrrg
SEQ ID NO: 8
Mouse NGFβ cDNA Sequence Variant 2
(NM_001112698.2, CDS from position
179 to 904)
      1 cagcacggca gagagcgcct ggagccggag
          gggagcgcat cgagttttgg cctgtggtcg
    61 tgcagtccag ggggctggat ggcatgctgg
          acccaagctc acctcagtgt ctgggcccaa
  121 taaaggtttt gccaaggacg cagctttcta
          tactggccgc agtgaggtgc atagcgtaat
  181 gtccatgttg ttctacactc tgatcactgc
          gtttttgatc ggcgtacagg cagaaccgta
  241 cacagatagc aatgtcccag aaggagactc
          tgtccctgaa gcccactgga ctaaacttca
  301 gcattccctt gacacagccc tccgcagagc
          ccgcagtgcc cctactgcac caatagctgc
  361 ccgagtgaca gggcagaccc gcaacatcac
          tgtagacccc agactgttta agaaacggag
  421 actccactca ccccgtgtgc tgttcagcac
          ccagcctcca cccacctctt cagacactct
  481 ggatctagac ttccaggccc atggtacaat
          ccctttcaac aggactcacc ggagcaagcg
  541 ctcatccacc cacccagtct tccacatggg
          ggagttctca gtgtgtgaca gtgtcagtgt
  601 gtgggttgga gataagacca cagccacaga
          catcaagggc aaggaggtga cagtgctggc
  661 cgaggtgaac attaacaaca gtgtattcag
          acagtacttt tttgagacca agtgccgagc
  721 ctccaatcct gttgagagtg ggtgccgggg
          catcgactcc aaacactgga actcatactg
  781 caccacgact cacaccttcg tcaaggcgtt
          gacaacagat gagaagcagg ctgcctggag
  841 gttcatccgg atagacacag cctgtgtgtg
          tgtgctcagc aggaaggcta caagaagagg
  901 ctgacttgcc tgcagccccc ttccccacct
          gccccctcca cactctcctg ggcccctccc
  961 tacctcagcc tgtaaattat tttaaattat
          aaggactgca tgataattta tcgtttatac
1021 aattttaaag acattattta ttaaattttc
          aaagcatcct gtataccga
SEQ ID NO: 9
Mouse NGFβ Amino Acid Sequence
Isoform B (NP_001106168.1)
      1 msmlfytlit afligvqaep ytdsnvpegd
          svpeahwtkl qhsldtalrr arsaptapia
    61 arvtgqtrni tvdprlfkkr rlhsprvlfs
          tqppptssdt idldfqahgt ipfnrthrsk
  121 rssthpvfhm gefsvcdsvs vwvgdkttat
          dikgkevtvl aevninnsvf rqyffetkcr
  181 asnpvesgcr gidskhwnsy cttthtfvka
          ittdekqaaw rfiridtacv cvlsrkatrr
241 g
SEQ ID NO: 10
Human hCG Amino Acid Sequence
Isoform 2 (NP_001305994)
      1 mggtwaskep Irprcrpina tlavekegcp
          vcitvnttic agycptmtrv iqgvlpalpq
    61 vvcnyrdvrf esiripgcpr gvnpvvsyav
          alscqcalcr rsttdcggpk dhpltcddpr
  121 fqasssskap ppslpspsrl pgpsdtpilp
          q
SEQ ID NO: 11
Human hCG Amino Acid Sequence
Isoform 1 (NP_203696)
      1 mskgllllll ismggtwask eplrprcrpi
          natlavekeg cpvcitvntt icagycptmt
    61 rvlqgvlpal pqvvcnyrdv rfesirlpgc
          prgvnpvvsy avalscqcal crrsttdcgg
  121 pkdhpltcdd prfqassssk apppslpsps
          rlpgpsdtpi ipq
SEQ ID NO: 12
Human hCG Amino Acid Sequence
Isoform 3 (NP_000728)
      1 memfqgllll lllsmggtwa skeplrprcr
          pinatlavek egcpvcitvn tticagycpt
    61 mtrvlqgvlp alpqvvcnyr dvrfesirlp
          gcprgvnpvv syavalscqc alcrrsttdc
  121 ggpkdhpltc ddprfqdsss skapppsips
          psrlpgpsdt pilpq
SEQ ID NO: 13
CTP of Human hCG Amino Acid Sequence
1 sssskapppsl pspsripgp sdtpilpq
```

Included in Table 1 are nucleic acid molecules comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with a nucleic acid sequence of SEQ ID NO: 1, 4, 6, and/or 8 listed in Table 1. Such nucleic acid molecules can encode a polypeptide having an NGF function described herein.
Included in Table 1 are polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of SEQ ID NO: 2, 3, 5, 7, and/or 9 listed in Table 1. Such polypeptides preferably have a NGF function described herein.

The therapeutic composition described herein may be administered, alone or in combination with a therapeutically acceptable carrier, to the subject through any suitable route. Such administration may be systemic (e.g., IV) or local (e.g., to a nerve or to the cerebrospinal fluid). A preferred administration route is parenteral (e.g., intravenous or injection). Without limitation, the administration of the NGF variants described herein can be done in a variety of ways, e.g., those routes known for specific indications, including, but not limited to, subcutaneously, intravenously, intracerebrally, intranasally, transdermally, intraperitoneally, intramuscularly, intrapulmonary, vaginally, rectally, intraarterially, intralesionally, intrathecally, intraventricularly in the brain, or intraocularly. The NGF variants may be administered continuously by infusion into the fluid reservoirs of the CNS, although bolus injection is acceptable, using any available techniques, such as pumps or implantation. In some instances, for example, in the treatment of wounds, the NGF variants may be directly applied as a solution or spray. Sustained release systems can be used. Generally, where the disorder permits, one should formulate and dose the NGF variant for site-specific delivery. Administration can be continuous or periodic. Administration can be accomplished by a constant- or programmable-flow implantable pump or by periodic injections.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

In some embodiments, vectors and/or host cells are further provided. One aspect of the present invention pertains to the use of vectors, preferably expression vectors, containing a nucleic acid encoding a biomarker listed in Table 2, or a portion or ortholog thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. In one embodiment, adenoviral vectors comprising a biomarker nucleic acid molecule are used.

The recombinant expression vectors of the present invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of the desired polypeptide in prokaryotic or eukaryotic cells. For example, a NGF variant polypeptide can be expressed in bacterial cells such as E. coli, insect cells (using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase. Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Examples of suitable yeast expression vectors include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Examples of suitable baculovirus expression vectors useful for insect cell hosts include the pAc series (Smith et al. (1983) Mol. Cell Biol. 3:2156-2165) and the pVL series (Lucklow and Summers (1989) Virology 170:31-39). Examples of suitable mammalian expression vectors include pCDM8 (Seed, B. (1987) Nature 329:840) and pMT2PC (Kaufman et al. (1987) EMBO J. 6:187-195).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters and/or regulatory sequences to promote gene expression in the vertebrate nervous system and the neural tissue are well-known in the art (see, for example, Timmusk et al. (1993) Neuron 10(3):475-489; Kaneko and Sueoka (1993) Proc Natl Acad Sci USA. 90(10): 4698-4702; Twyman and Jones (1995) J. Neurogenetics 10:67-101).

The present invention further provides a recombinant expression vector comprising a nucleic acid molecule of the invention cloned into the expression vector in an antisense orientation. That is, the DNA molecule is operatively linked to a regulatory sequence in a manner which allows for expression (by transcription of the DNA molecule) of an RNA molecule which is antisense to a biomarker mRNA described herein. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, biomarker protein can be expressed in bacterial cells such as E. coli, insect cells, yeast or mammalian cells (such as Fao hepatoma cells, primary hepatocytes, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A cell culture includes host cells, media and other byproducts. Suitable media for cell culture are well known in the art. A biomarker polypeptide or fragment thereof, may be secreted and isolated from a mixture of cells and medium containing the polypeptide. Alternatively, a biomarker polypeptide or fragment thereof, may be retained cytoplasmically and the cells harvested, lysed and the protein or protein complex isolated. A biomarker polypeptide or fragment thereof, may be isolated from cell culture medium, host cells, or both using techniques known in the art for purifying proteins, including ion-exchange chromatography, gel filtration chromatography, ultrafiltration, electrophoresis, and inmmunoaffinity purification with antibodies specific for particular epitopes of a biomarker or a fragment thereof. In other embodiments, heterologous tags can be used for purification purposes (e.g., epitope tags and FC fusion tags), according to standards methods known in the art.

Thus, a nucleotide sequence encoding all or a selected portion of a biomarker polypeptide may be used to produce a recombinant form of the protein via microbial or eukaryotic cellular processes. Ligating the sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant biomarker polypeptides, or fragments thereof, by microbial means or tissue-culture technology in accord with the subject invention.

A host cell of the invention, such as a prokaryotic or eukaryotic host cell in culture, can be used to produce (i.e., express) biomarker protein. Accordingly, the invention further provides methods for producing biomarker protein using the host cells of the invention. In one embodiment, the method comprises culturing the host cell of invention (into which a recombinant expression vector encoding a biomarker has been introduced) in a suitable medium until biomarker protein is produced. In another embodiment, the method further comprises isolating the biomarker protein from the medium or the host cell.

### II. Subjects

In certain embodiments, the subject suitable for the compositions and methods disclosed herein is a mammal (e.g., mouse, rat, primate, non-human mammal, domestic animal, such as a dog, cat, cow, horse, and the like), and is preferably a human. In other embodiments, the subject is an animal model of metabolic disturbance or intolerance.

In other embodiments of the methods of the present invention, the subject has not undergone treatment for the disease or disorder. In still other embodiments, the subject has undergone treatment for the disease or disorder.

The methods of the present invention can be used to treat and/or determine the responsiveness to a composition described herein, alone or in combination with other therapies to achieve weight loss, in subjects such as those described herein.

### III. Pharmaceutical Compositions

The present invention provides pharmaceutically acceptable compositions of the compositions disclosed herein. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment, eye drops or spray applied to the skin or ocular administration; (4) intravaginally or intrarectally, for example, as a pessary, cream or foam; or (5) aerosol, for example, as an aqueous aerosol, liposomal preparation or solid particles.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Alternatively or additionally, compositions can be formulated for delivery via a catheter, stent, wire, or other intraluminal device. Delivery via such devices may be especially useful for delivery to the bladder, urethra, ureter, rectum, or intestine.

Ophthalmic formulations, eye ointments, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of ocular administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Injectable depot forms are made by forming microencapsulated matrices of the subject agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of administration may also include rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

In certain embodiments, NGF variants of the invention may be used alone or conjointly administered with another type of therapeutic agent.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In certain embodiments, the present invention also provides gene therapy for the in vivo production of the NGF variants described herein. Such therapy would achieve its therapeutic effect by introducing nucleic acids encoding the NGF variants described herein into cells or tissues having the disorders as listed above. Delivery of the nucleic acids described herein can be achieved using a recombinant expression vector such as a chimeric virus or a colloidal dispersion system. Targeted liposome may also be used for therapeutic delivery of the NGF variants described herein.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the NGF variants described herein. In a preferred embodiment, the vector is targeted to the nervous system or any cell, tissue, or organ having deficient wild type NGF levels and/or defective NGF mutants.

Alternatively, cultured cells can be directly transfected with plasmids encoding the retroviral structural genes gag, pol and env, by conventional calcium phosphate transfection. These cells can then be transfected with a vector plasmid containing the genes of interest. The resulting cells release the retroviral vector into the culture medium.

### EXAMPLES

### Example 1: Preparation of a Long-Lasting Recombinant Human Nerve Growth Factor (rhNGF)

Cloning of NGF-CTP

NGF (238 amino acids) is initially a complex of α-NGF, β-NGF and γ-NGF, and the γ subunit cleaves the N-terminal of the β subunit, thereby activating the protein into functional NGF (120 amino acids). The full-length NGF gene (714-bp) was amplified with the plasmid that has the full-length human NGF gene, using 5′-ATCTC GAGCA CCATG TCCAT GTTGT TCTAC ACTCT GA-3′ (SEQ ID NO:14) as the forward primer (U1), and 5′-TGGAG CCTTG GAAGA GCTAG AGGCT CTTCT CACAG CCTT-3′ (SEQ ID NO:15) as the reverse primer (R1). The CTP gene from human HCG was synthesized by Sangon Biotech (Shanghai) Co., Ltd., and the forward (U2) and reverse (R2) primers designed to amplify this gene are 5′-AAGGC TGTGA GAA GA GCCTC TAGCT CTTCC AAGGC TCCA-3′ (SEQ ID NO:16) and 5′-TTTGC GGCCG CTTACT ACTGG GGCAG AATA-3′ (SEQ ID NO:17), respectively. The NGF sequence and the CTP sequence were thus amplified and analyzed with gel electrophoresis, followed by gel extraction to obtain the PCR products. PCR overlap extension was performed using the PCR products of NGF (1 μL) and HCG CTP (1 μL) as the templates, and U1 and R2 as the forward and reverse primers, respectively. PCR amplification consisted of an initialization step (94° C., 3 min), a denaturation step (94° C., 30 sec), an annealing step (58° C., 30 sec) and an elongation step (72° C., 1 min) for 30 cycles, followed by a final elongation step (72° C., 7 min). The PCR product was analyzed with gel electrophoresis and then extracted. The gene from gel extraction was ligated to pMC-18T vector and then positive clones were screened and selected for further gene sequencing. The clones with the correct sequences were saved for further constructions. The gene sequence is shown in SEQ ID NO:1, and the amino acid sequence in SEQ ID NO: 2 (Table 1).

Construction of NGF-CTP Expression Factor

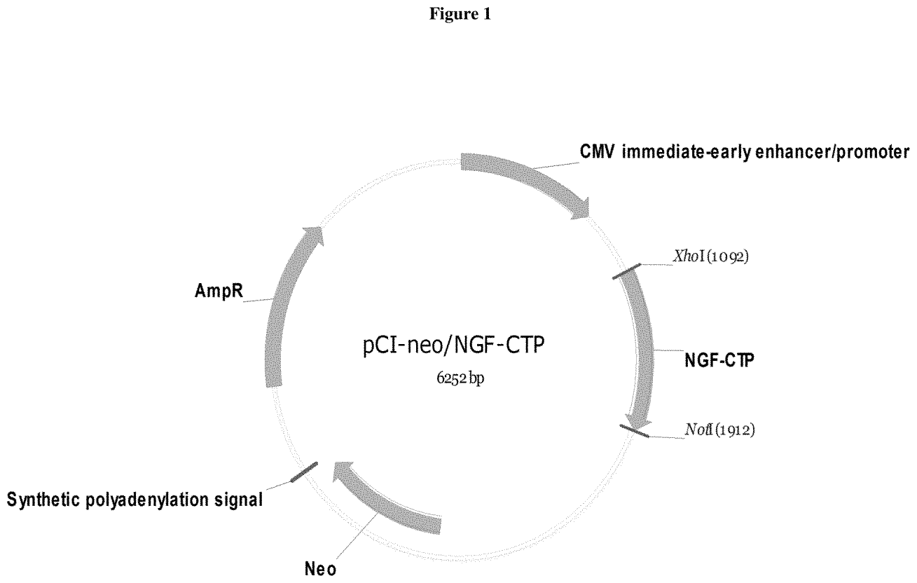

The NGF-CTP fragment was derived by cleavage of the pMC-18T/NGF-CTP plasmid with XhoI and NotI (New England Biolabs Ltd.) as the restriction enzymes. The fragment was then ligated to the pCI-neo vector, which was pre-treated with XhoI and NotI. After the ligation reaction at 16° C. overnight, the product was transformed to DH5a competent cells, and screened on LB-agar plates with ampicillin. The positive clones were first verified by PCR, and further assessed by gene sequencing. The construction of the PCI-neo/NGF-CTP plasmid is shown in FIG. 1.

Construction of the Mammalian Cell Expression System for rhNGF-CTP

The PCI-neo/NGF-CTP plasmid was introduced to CHO-S cells (Invitrogen Co.) through electroporation, using Gene Pulser Xcell™ electroporation system (Bio-Rad Laboratories, Inc.) at 160 V for 150 ms. The electroporated cells were transferred to and cultured on a 35-mm tissue culture dish with DMEM-F12 culture media supplemented with 10% fetal bovine serum (FBS). Two days later, G418 (Sigma-Aldrich Co. LLC.) was added to the culture media to a final concentration of 600 μg mL⁻¹ to enhance the selection for the resistant gene. The remaining monoclonal cells after G418 treatment were transferred to 96-well plates for further analysis of the protein expression level via dot blotting. The cells with high expression level were selected for subsequent suspension culture.

After suspension culture, the cells with the highest expression level were transferred to tissue culture flasks (40 mL, Corning Inc.) for an additional selection of the ones that are productive under serum-free conditions. The cells were cultured with CD CHO media (Invitrogen Co.) at 37° C., and the cell growth was evaluated, as well as the expression level of the recombinant protein assayed by ELISA (R&D Systems, Inc.). An additional subclone was used to confirm the recombinant 1B2 cells are the prototype cell line of the rhNGF-CTP production. This cell line was extensively tested for sterility and contaminants (e.g. bacteria and mycoplasmas), before developing the master cell bank and the working cell bank for rhNGF-CTP expression.

Purification of rhNGF-CTP

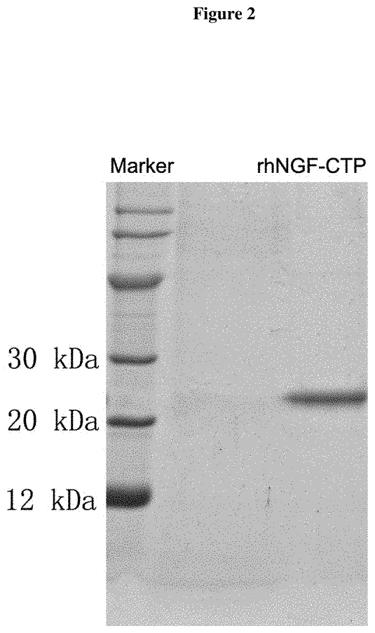

A working cell bank was recovered to produce rhNGF-CTP with serum-free culture media in a WAVE bioreactor (10 L, GE Healthcare). The cells were cultured in the bioreactor with the Fed Batch mode for 12 days, before being harvested. The supernatant was collected, concentrated with centrifugal filters, and purified with protein chromatography on an ÄKTA purifier (GE Healthcare). The sample was first applied to Sepharose Fastflow (GE Healthcare), eluted by buffer with sodium chloride (1 mol L⁻¹). And then Phenyl Fastflow (GE Healthcare) and Superdex 75 (GE Healthcare) were used for further purification. The purity of the recombinant protein was over 95% as shown in the SDS-PAGE gel electrophoresis (FIG. 2).

Characterization of rhNGF-CTP and its Biological Activity

N-terminal sequencing of the purified rhNGF-CTP aligned well with the gene sequence of the human NGF, with the same first five amino acids (SSSHP), which indicated the correct cleavage of α-NGF. The sequence of the rhNGF-CTP (148 amino acids) is shown in SEQ ID No. 3. The calculated molecular weight of the non-glycosylated rhNGF-CTP is 16273 Da, and the molecular weight of the purified rhNGF-CTP is 18605 Da, measured by mass spectrometry. The difference in the molecular weights is attributed to glycosylation on the CTP.

The biological activity of rhNGF-CTP was then investigated by two cell assays. First, TF-1 cell proliferation assay was used to evaluate the dose-dependent stimulation of TF-1 cell growth induced by NGF, which functions through binding with the high-affinity TrkA receptor on the TF-1 cell surface. The biological activity of NGF was calculated through the cell proliferation rate (MTT assay) of the stimulated TF-1 cells. The rhNGF standard was the NGF sample from National Institute for Biological Standards and Control (NIBSC) of UK, with the specific activity of 1×106 AU mg⁻¹. The specific activity of rhNGF-CTP and the NGF standard in the TF-1 cell proliferation assay was 1.2×10⁶ AU mg⁻¹ and 1.8×10⁶ AU mg⁻¹, respectively, with no significant difference observed. A second method that measures the NGF activity semi-quantitatively was the sprouting of the embryonic chicken dorsal root ganglion (DRG). The mouse NGF from National Institutes for Food and Drug Control of China was used as the standard sample here. The specific activity of rhNGF-CTP and the NGF standard was ≥1.7×10⁵ AU mg⁻¹ and ≥5×10⁵ AU mg⁻¹, respectively. A slight decrease of the biological activity was seen for rhNGF-CTP.

The Pharmacokinetics Study of rhNGF-CTP in Rats

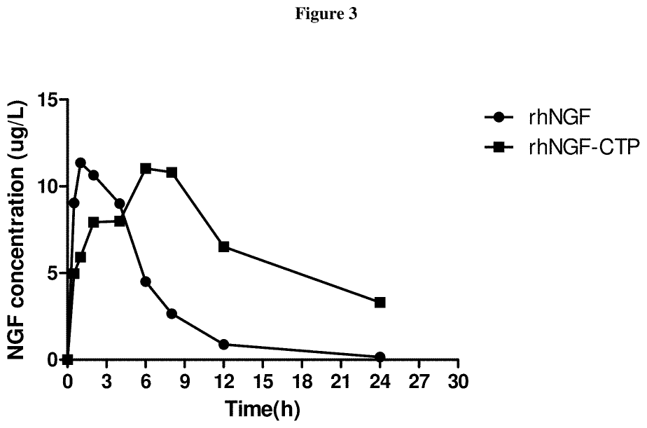

Sprague Dawley® rats were subjected to the pharmacokinetics study of rhNGF-CTP. Six rats (body weight 300-400 g) were separated into two groups, treated with either rhNGF or rhNGF-CTP. The rats were anesthetized with napental (1%), and rhNGF or rhNGF-CTP were administrated through intramuscular injections at 30 μg kg⁻¹ body weight. Blood samples were collected from the tail at 0.5, 1, 2, 4, 6, 8, 12, and 24 hr. after injection. The plasma NGF concentration was determined with ELISA, as shown in FIG. 3. The half-life of rhNGF was calculated to be 3.9 hr. in rats, whereas that of rhNGF-CTP extended to 10.0 hr., which indicated a 2.5-fold increase.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1-60. (canceled)

61. A polypeptide comprising:
i) first portion comprising a full-length nerve growth factor (NGF) polypeptide, or a biologically active fragment thereof, wherein the first portion comprises the amino acid sequence of residues 122 to 241 of SEQ ID NO: 5; and
ii) a second portion comprising an additional polypeptide that increases the half-life of the NGF polypeptide, or biologically active fragment thereof, in a bloodstream of a human, wherein the second portion comprises the amino acid sequence of SEQ ID NO: 13,
wherein the polypeptide has an in vivo half-life at least 2.5 times the in vivo half-life of the NGF polypeptide alone.

62. The polypeptide of claim 61, wherein the first portion comprises a full-length NGF polypeptide sequence.

63. The polypeptide of claim 61, wherein the first portion comprises a biologically active fragment of NGF.

64. The polypeptide of claim 61, wherein the first portion comprises a human NGF sequence.

65. The polypeptide of claim 61, wherein the NGF polypeptide comprises an NGF sequence that is at least 99% identical to SEQ ID NO: 5.

66. The polypeptide of claim 64, wherein the human NGF sequence comprises SEQ ID NO: 5.

67. The polypeptide of claim 63, wherein the first portion is capable of binding to at least one binding partner of NGF, optionally wherein the at least one binding partner of NGF is a tropomyosin receptor kinase A (TrkA) or a low-affinity NGF receptor (LNGFR/p75NTR).

68. The polypeptide of claim 67, wherein the first portion consists of amino acid residues from position 122 to position 241 of SEQ ID NO: 5.

69. The polypeptide of claim 61, wherein the second portion comprises a full-length human chorionic gonadotropin (HCG), or a biologically active fragment thereof.

70. The polypeptide of claim 69, wherein the human chorionic gonadotropin (HCG) comprises an amino acid sequence selected from SEQ ID NOs: 10-12.

71. The polypeptide of claim 61, wherein the second portion comprises a carboxyl-terminal portion (CTP) of HCG.

72. The polypeptide of claim 61, wherein the second portion consists of an amino acid sequence selected from SEQ ID NOs:10-12.

73. The polypeptide of claim 61, wherein the second portion consists of the amino acid sequence of SEQ ID NO:13.

74. The polypeptide of claim 61, wherein the second portion comprises at least one glycosylation site.

75. The polypeptide of claim 61, wherein the first portion and the second portion are fused together with a linker.

76. The polypeptide of claim 61, consisting of the amino acid sequence of SEQ ID NO: 2 or 3.

77. The polypeptide of claim 61, further comprising a third portion comprising a fusion domain that increases the function and/or stability of the NGF polypeptide sequence, or biologically active fragment thereof, in the bloodstream of a human.
